SEQUENCE LISTING (1 sequence)

SEQ ID NO: 1 - protein, 4 aa, Artificial Sequence
  synthetic DEAH box
DEAH

(19) United States
(12) Patent Application Publication
(10) Pub. No.: US 2013/0274128 A1
(43) Pub. Date: Oct. 17, 2013

(54) GENE EXPRESSION IN N-CADHERIN OVEREXPRESSING PROSTATE CANCERS AND THEIR CONTROLS

(71) Applicant: Robert E. Reiter, Los Angeles, CA (US)

(72) Inventor: Robert E. Reiter, Los Angeles, CA (US)

(73) Assignee: The Regents of the University of Califorina, Oakland, CA (US)

(21) Appl. No.: 13/825,100

(22) PCT Filed: Sep. 20, 2011
(86) PCT No.: PCT/US11/52393

§ 371 Date: Jun. 28, 2013

Related U.S. Application Data
(60) Provisional application No. 61/385,112, filed on Sep. 21, 2010.
(60) Provisional application No. 61/385,438, filed on Sep. 22, 2010.
(60) Provisional application No. 61/514,383, filed on Aug. 2, 2011.

(30) Foreign Application Priority Data
Feb. 1, 2011 (US) PCT/US2011/023407

(51) Int. Cl. C12Q 1/68; G01N 33/50

(52) U.S. Cl. CPC C12Q 1/6886; G01N 33/5091
USPC 506/9; 435/6.14; 506/2; 435/6.12; 435/7.23; 435/7.4; 435/7.92; 435/34; 435/6.11

(57) ABSTRACT

The present invention provides methods of diagnosing a cancer or providing a prognosis for a cancer by analyzing the level of expression of a marker that is a downstream target of N-cadherin.

### CROSS-REFERENCES TO RELATED APPLICATIONS

This application relates to U.S. Provisional Patent Application Ser. No. 61/385,112, filed on Sep. 21, 2010, and to U.S. Provisional Patent Application Ser. No. 61/385,438, filed on Sep. 22, 2010, and International Patent Application Serial No. PCT/US2011/023407 filed on Feb. 1, 2011, and U.S. Provisional Patent Application Ser. No. 61/514,383 filed on Aug. 2, 2011, the contents of each of which are incorporated herein by reference in its entirety.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Prostate cancer is the most common non-skin cancer in the United States, affecting 1 in 6 men. Prostate cancer is a biologically and clinically heterogeneous disease. A majority of men with this malignancy harbor slow-growing tumors that may not impact an individual's natural lifespan, while others are struck by rapidly progressive, metastatic tumors. PSA screening is limited by a lack of specificity and an inability to predict which patients are at risk to develop hormone refractory metastatic disease. Studies advocating a lower PSA threshold for diagnosis may increase the number of prostate cancer diagnoses and further complicate the identification of patients with indolent vs. aggressive cancers (Punglia et al., N Engl J Med, 349:335-342 (2003)). New serum and tissue markers that correlate with clinical outcome or identify patients with potentially aggressive disease are urgently needed (Welsh et al., Proc Natl Acad Sci USA, 100:3410-3415 (2003)).

In order to identify new candidate serum or tissue markers of hormone refractory prostate cancer, we have previously compared gene expression profiles of paired hormone dependent and hormone refractory prostate cancer xenografts. The LAPC-9 xenograft was established from an osteoblastic bone metastasis and progresses from androgen dependence to independence following castration in immune deficient mice (Craft et al., Cancer Research, 59:5030-6 (1999)). It has been used previously to identify candidate therapeutic targets in prostate cancer. Differentially expressed genes were validated and then examined for sequence homology to secreted or cell surface proteins. N-cadherin has been identified as a marker of cancer. The identification, characterization and initial validation of N-cadherin, which is expressed in both hormone refractory prostate cancer and bladder cancer, has been previously reported (see WO 2007/109347, the contents of which are hereby incorporated by reference in its entirety). Recent studies in our laboratory have shown that N-cadherin is upregulated in a large percentage of advanced prostate cancers.

One type of cell movement than can be observed in embryogenesis requires the loss of cell-cell contacts for the migration of individual cells or small group of cells through the extracellular matrix. This process is called epithelial to mesenchymal transition (EMT). EMT also occurs in pathological situations, such as the acquisition of a motile and invasive phenotype of tumor cells of epithelial origin. A hallmark of EMT is the loss of E-cadherin and the de novo expression of N-cadherin adhesion molecules. N-cadherin promotes tumor cell survival, migration and invasion, and high levels of N-cadherin expression is often associated with poor prognosis. N-cadherin is also expressed in endothelial cells and plays an essential role in the maturation and stabilization of normal vessels and tumor-associated angiogenic vessels.

N-cadherin and associated EMT are common features not only of prostate cancer but also other solid malignancies such as bladder cancer and melanoma. Thus, downstream targets of N-cadherin which are associated with EMT are potentially valuable diagnostic and therapeutic targets in cancer. Accordingly, the present invention provides methods which target downstream targets of N-cadherin in the diagnosis, prognosis, and treatment of cancers expressing N-cadherin, including but not limited to prostate cancer.

### BRIEF SUMMARY OF THE INVENTION

In one aspect, the present invention provides methods of diagnosing a cancer in a subject. In some embodiments, the method comprises:
(a) analyzing a tissue sample from the subject with an assay that specifically detects at least one marker that is a downstream target of N-cadherin, wherein the at least one marker is selected from the markers listed in Table 2 or Table 3 or Table 4; and
(b) determining whether or not expression of the at least one marker is altered in the tissue sample; thereby providing a diagnosis for the cancer.

In some embodiments, step (b) comprises determining whether or not the at least one marker is overexpressed in the tissue sample; thereby providing the diagnosis for the cancer. In some embodiments of the above, step (a) further comprises analyzing a marker listed in Table 1 and step (b) further comprises determining whether or not expression of the marker from Table 1 is also altered or overexpressed in the tissue sample. In some embodiments, the method involves analyzing of set of genes which include a different marker from each of Tables 1, 2, 3, and 4. In some embodiments, the method analyzes a set of markers which include a marker from each of Table 2, 3 or 4 which is not found in Table 1.

In another aspect, the present invention provides methods of providing a prognosis for a cancer in a subject. In some embodiments, the method comprises:
(a) analyzing a tissue sample from the subject with an assay that specifically detects at least one marker that is a downstream target of N-cadherin, wherein the at least one marker is selected from the markers listed in Table 2 or Table 3 or Table 4; and
(b) determining whether or not expression of the at least one marker is altered in the tissue sample; thereby providing a prognosis for the cancer.

In some embodiments, step (b) comprises determining whether or not the at least one marker is overexpressed in the tissue sample; thereby providing the prognosis for the cancer. In some embodiments of the above, step (a) further comprises analyzing a marker listed in Table 1 and step (b) further comprises determining whether or not expression of the marker from Table 1 is also altered or overexpressed in the tissue sample. In some embodiments, the method analyzes a set of markers which include a marker from each of Table 2, 3 or 4 which is not found in Table 1. In some embodiments, the method involves analyzing of set of genes which include a different marker from each of Tables 1, 2, 3, and 4.

In some embodiments, the assay detects nucleic acid and is mass spectroscopy, PCR, microarray hybridization, thermal cycle sequencing, capillary array sequencing, or solid phase sequencing. In some embodiments, the assay detects protein and is ELISA, Western blotting, flow cytometry, immunofluorescence, immunohistochemistry, or mass spectroscopy.

In some embodiments, the assay comprises a reagent that binds to a nucleic acid. In some embodiments, the reagent is a nucleic acid. In some embodiments, the reagent is an oligonucleotide. In some embodiments, the reagent is an RT-PCR primer set.

In some embodiments, the assay comprises a reagent that binds to a protein. In some embodiments, the reagent is an antibody.

In some embodiments, the cancer is an N-cadherin-expressing cancer. In some embodiments, the cancer is prostate cancer.

In some embodiments, the at least one marker is selected from FIG. 4.

In some embodiments, the tissue sample is a metastatic cancer tissue sample. In some embodiments, the tissue sample is prostate tissue.

### BRIEF DESCRIPTION OF THE DRAWINGS

FIG. 1. A-D. RT-PCT analysis confirming differential expression of candidate genes in LNCaP cell lines FGC (control), C1 (high expressing N-cadherin line), C2 (intermediate expressing N-cadherin line), C3 (low expressing N-cadherin line), and CL-1 (an endogenous N-cadherin expressing LNCaP cell line).

FIG. 2. Western blot analysis confirming upregulation of axl kinase in LNCaP cell lines C1, C2, and CL-1.

FIG. 3. A-D. Western blots of normal and malignant primary prostate cancers for selected candidate genes, including 9 genes in which the limited samples used confirmed an association of the specific gene with prostate cancer (either higher expression in cancer vs. normal, or expression only in cancer or high grade cancer) (D).

FIG. 4. Genes of particular interest which are up-regulated in prostate cancers which overexpress N-cadherin.

### DETAILED DESCRIPTION OF THE INVENTION

### I. Introduction

The present invention relates to markers that are downstream targets of N-cadherin which have altered expression levels in cancer tissues. N-cadherin is found on cell surfaces, expressed in many epithelial tumors, and is associated with invasion, metastasis, EMT, and possibly androgen independence. N-cadherin is overexpressed in a large percentage of advanced prostate cancers as well as in other malignancies such as bladder cancer and melanoma. The markers described herein are upregulated in cancer tissues, including N-cadherin-overexpressing cancer tissues. These markers are therefore useful diagnostic and prognostic targets as well as useful targets for therapeutic intervention. To our knowledge, an approach to diagnostic or therapeutic target discovery by looking at downstream targets of N-cadherin has not been undertaken previously.

The invention also relates to methods of diagnosing or providing a prognosis for cancers expressing N-cadherin or exhibiting EMT by detecting the expression levels of any of the markers that are downstream targets of N-cadherin as described herein (e.g., a marker listed in Table 1 or Table 2). Generally, the methods find use in diagnosing or prognosing a cancer such as a urogenital cancer (e.g., prostate cancer or bladder cancer). For diagnostic and prognostic methods, either protein or mRNA can be detected. The markers of the present invention can be measured by techniques such as RT-PCR, microarray, Western, ELISA, etc. Any specific probe can be used for detection, such as an antibody, a receptor, a ligand, RT-PCR etc. The diagnostic and prognostic methods may detect a single marker that is a downstream target of N-cadherin, or may detect two or more markers that are downstream targets of N-cadherin.

The invention further relates to methods of treating a cancer expressing N-cadherin or exhibiting EMT by targeting at least one marker that is a downstream target of N-cadherin (e.g., at least one marker listed in Table 1 or Table 2 or Table 3). For therapeutic methods, any antibody or inhibitory oligonucleotide (e.g., RNAi, siRNA, aptamers, ribozymes, etc.) can be used to target the marker and thus treat the cancer.

### II. Definitions

“N-cadherin” refers to nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region at least about 25, 50, 100, 200, 500, 1000, or more amino acids, to a polypeptide encoded by a respectively referenced nucleic acid or an amino acid sequence described herein, for example, as depicted in GenBank Accession Nos. NM_{—}001792 (N-Cadherin mRNA) and NP_{—}001783 (N-Cadherin protein); (2) specifically bind to antibodies, e.g., polyclonal antibodies, raised against an immunogen comprising a referenced amino acid sequence as depicted in GenBank Accession No. NP_{—}001783 (N-Cadherin protein); immunogenic fragments respectively thereof, and conservatively modified variants respectively thereof; (3) specifically hybridize under stringent hybridization conditions to a nucleic acid encoding a referenced amino acid sequence as depicted in GenBank Accession No. NP_{—}001783 (N-Cadherin protein) and conservatively modified variants respectively thereof; (4) have a nucleic acid sequence that has greater than about 95%, preferably greater than about 96%, 97%, 98%, 99%, or higher nucleotide sequence identity, preferably over a region of at least about 25, 50, 100, 150, 200, 250, 500, 1000, or more nucleotides, to a reference nucleic acid sequence as shown in GenBank Accession No. NM_{—}001792 (N-Cadherin mRNA). A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules.

The term “marker” refers to a molecule (e.g., protein nucleic acid) that is expressed in the cell, expressed on the surface of a cancer cell or secreted by a cancer cell in comparison to a normal cell, and which is useful for the diagnosis of cancer, or for providing a prognosis. Such markers are molecules that are differentially expressed, e.g., overexpressed or underexpressed in a prostate cancer tissue or other cancer tissue in comparison to a normal tissue or in an N-cadherin-overexpressing prostate cancer tissue or other cancer tissue in comparison to a non-N-cadherin-overexpressing cancer tissue, for instance, 1-fold over/under expression, 2-fold over/under expression, 3-fold over/under expression or more in comparison to a normal tissue or non-N-cadherin-overexpressing cancer tissue.

It will be understood by the skilled artisan that markers may be used singly or in combination with other markers for any of the uses, e.g., diagnosis or prognosis of prostate cancer, as disclosed herein.

The term “downstream target,” when used in the context of a downstream target of N-cadherin, refers to a gene or protein whose expression is directly or indirectly regulated by N-cadherin. In some embodiments, a downstream target is a gene or protein whose expression is upregulated, directly or indirectly, by N-cadherin. In some embodiments, a downstream target is a gene or protein whose expression is downregulated, directly or indirectly, by N-cadherin. In some embodiments, a downstream target of N-cadherin is a marker listed in Table 1 or Table 2 or Table 3 infra.

“Cancer” refers to human cancers and carcinomas, sarcomas, adenocarcinomas, lymphomas, leukemias, etc., including solid tumors and lymphoid cancers, kidney, breast, lung, kidney, bladder, colon, ovarian, prostate, pancreas, stomach, brain, head and neck, skin, uterine, testicular, esophagus, and liver cancer, lymphoma, including non-Hodgkin's and Hodgkin's lymphoma, leukemia, and multiple myeloma. “Urogenital cancer” refers to human cancers of urinary tract and genital tissues, including but not limited to kidney, bladder, urinary tract, urethra, prostrate, penis, testicle, vulva, vagina, cervical and ovary tissues. In some embodiments, the cancer to be diagnosed, prognosed, or treated herein is characterized by excessive activation of N-cadherin.

The terms “overexpress,” “overexpression,” or “overexpressed” interchangeably refer to RNA or protein expression of a marker of interest in a prostate cancer tissue or other cancer tissue sample that is detectably higher than RNA or protein expression of the marker of interest in a control tissue sample. Overexpression can be due to increased transcription, post transcriptional processing, translation, post translational processing, altered stability, or altered protein degradation, as well as local overexpression due to altered protein traffic patterns (increased nuclear localization), and augmented functional activity, e.g., as a transcription factor. Overexpression can be detected using conventional techniques for detecting mRNA (e.g., RT-PCR, PCR, microarray) or proteins (e.g., ELISA, Western blots, flow cytometry, immunofluorescence, immunohistochemistry, DNA binding assay techniques). Overexpression can be 10%, 20%, 30%, 40%, 50%, 60%, 70%, 80%, 90% or more for the marker of interest in the prostate cancer tissue or other cancer tissue sample in comparison to a control (e.g., non-cancer) tissue. In certain instances, overexpression is 1-fold, 2-fold, 3-fold, 4-fold or more higher levels of RNA or protein levels for the marker of interest in the prostate cancer tissue or other cancer tissue sample in comparison to a control (e.g., non-cancer) tissue.

The terms “underexpress,” “underexpression,” or “underexpressing” interchangeably refer to RNA or protein expression of a marker of interest in a prostate cancer tissue or other cancer tissue sample that is detectably lower than RNA or protein expression of the marker of interest in a control tissue sample. Underexpression can be due to decreased transcription, post transcriptional processing, translation, post translational processing, altered stability, or altered protein degradation, as well as local underexpression due to altered protein traffic patterns (increased nuclear localization), and augmented functional activity, e.g., as an enzyme. Underexpression can be detected using conventional techniques for detecting mRNA (e.g., RT-PCR, PCR, microarray) or proteins (e.g., ELISA, Western blots, flow cytometry, immunofluorescence, immunohistochemistry, DNA binding assay techniques). Underexpression can be 90%, 80%, 70%, 60%, 50%, 40%, 30%, 20%, 10% or less for the marker of interest in the prostate cancer tissue or other cancer tissue sample in comparison to a control (e.g., non-cancer) tissue. In certain instances, underexpression is 1-fold, 2-fold, 3-fold, 4-fold or more lower levels of RNA or protein levels for the marker of interest in the prostate cancer tissue or other cancer tissue sample in comparison to a control (e.g., non-cancer) tissue.

“Biological sample” includes sections of tissues such as biopsy and autopsy samples, and frozen sections taken for histological purposes. Such samples include blood and blood fractions or products (e.g., serum, plasma, platelets, red blood cells, and the like), sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, or mouse; rabbit; bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (i.e., prostate, lymph node, liver, bone marrow, blood cell), the size and type of the tumor (i.e., solid or suspended (i.e., blood or ascites)), among other factors. Representative biopsy techniques include excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

The terms “identical” or “percent identity,” in the context of two or more nucleic acids or polypeptide sequences, refer to two or more sequences or subsequences that are the same or have a specified percentage of amino acid residues or nucleotides that are the same (i.e., at least 60% identity, at least 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant,” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous,” when used with reference to portions of a nucleic acid, indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

Accordingly, the term antibody also embraces minibodies, diabodies, triabodies and the like. Diabodies are small bivalent biospecific antibody fragments with high avidity and specificity. Their high signal to noise ratio is typically better due to a better specificity and fast blood clearance increasing their potential for diagnostic and therapeutic targeting of specific antigen (Sundaresan et al., J Nucl Med 44:1962-9 (2003). In addition, these antibodies are advantageous because they can be engineered if necessary as different types of antibody fragments ranging from a small single chain Fv to an intact IgG with varying isoforms (Wu & Senter, Nat.Biotechnol. 23:1137-1146 (2005)). In some embodiments, the antibody fragment is part of a diabody.

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In some embodiments, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

“RNAi molecule” or an “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferably about preferably about 20-30 base nucleotides, preferably about 20-25 nucleotides in length, e.g., 20, 21, 22, 23, 24, 25, 26, 27, 28, 29, or 30 nucleotides in length).

An “antisense” polynucleotide is a polynucleotide that is substantially complementary to a target polynucleotide and has the ability to specifically hybridize to the target polynucleotide. An antisense polynucleotide for use in the present invention can be one which specifically hybridizes to a polynucleotide of a marker that is a downstream target of N-cadherin, e.g., a marker listed in Table 1 or Table 2 or Table 3.

“Aptamers” are DNA or RNA molecules that have been selected from random pools based on their ability to bind other molecules with high affinity specificity (see, e.g., Cox and Ellington, Bioorg. Med. Chem. 9:2525-2531 (2001); Lee et al., Nuc. Acids Res. 32:D95-D100 (2004)). Aptamers have been selected which bind nucleic acid, proteins, small organic compounds, vitamins, inorganic compounds, cells, and even entire organisms. An aptamer for use in the present invention can be one which binds with high affinity (e.g., having a K_{d} of less than 100 nM, 10 nM, or 1 nM) to a marker that is a downstream target of N-cadherin, e.g., a marker listed in Table 1 or Table 2 or Table 3.

“Ribozymes” are enzymatic RNA molecules capable of catalyzing specific cleavage of RNA. The composition of ribozyme molecules preferably includes one or more sequences complementary to a target mRNA, and the well known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave target mRNA transcripts can also be used to prevent translation of subject target mRNAs.

“Inhibitors,” “activators,” and “modulators” of the markers are used to refer to activating, inhibitory, or modulating molecules identified using in vitro and in vivo assays of the markers that are downstream targets of N-cadherin. “Inhibitors” are compounds that, e.g., bind to, partially or totally block activity, decrease, prevent, delay activation, inactivate, desensitize, or down regulate the activity or expression of the markers that are downstream targets of N-cadherin. “Activators” are compounds that increase, open, activate, facilitate, enhance activation, sensitize, agonize, or up regulate activity of the markers that are downstream targets of N-cadherin, e.g., agonists. Inhibitors, activators, or modulators also include genetically modified versions of the markers, e.g., versions with altered activity, as well as naturally occurring and synthetic ligands, antagonists, agonists, antibodies, peptides, cyclic peptides, nucleic acids, antisense molecules, ribozymes, RNAi molecules, small organic molecules and the like. Such assays for inhibitors and activators include, e.g., expressing the markers that are downstream targets of N-cadherin in vitro, in cells, or cell extracts, applying putative modulator compounds, and then determining the functional effects on activity.

The phrase “functional effects” in the context of assays for testing compounds that modulate a marker that is a downstream target of N-cadherin includes the determination of a parameter that is indirectly or directly under the influence of a biomarker of the invention, e.g., a chemical or phenotypic. A functional effect therefore includes ligand binding activity, transcriptional activation or repression, the ability of cells to proliferate, the ability to migrate, among others. “Functional effects” include in vitro, in vivo, and ex vivo activities.

By “determining the functional effect” is meant assaying for a compound that increases or decreases a parameter that is indirectly or directly under the influence of a biomarker of the invention, e.g., measuring physical and chemical or phenotypic effects. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of the marker; measuring changes in enzymatic activity; the ability to increase or decrease cellular proliferation, apoptosis, cell cycle arrest, measuring changes in cell surface evaluated by many means known to those skilled in the art, e.g., microscopy for quantitative or qualitative measures of alterations in morphological features, measurement of changes in RNA or protein levels for other genes expressed in placental tissue, measurement of RNA stability, identification of downstream or reporter gene expression (CAT, luciferase, β-gal, GFP and the like), e.g., via chemiluminescence, fluorescence, colorimetric reactions, antibody binding, inducible markers, etc.

Samples or assays comprising markers that are downstream targets of N-cadherin that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a marker is achieved when the activity value relative to the control is about 80%, preferably 50%, more preferably 25-0%. Activation of a marker is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, peptide, circular peptide, lipid, fatty acid, siRNA, polynucleotide, oligonucleotide, etc., to be tested for the capacity to directly or indirectly modulate a marker as described herein. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e g, inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

### III. Diagnostic and Prognostic Methods

The present invention provides methods of diagnosing a cancer in a subject. As used herein, the term “diagnosing” or “diagnosis” refers to detecting a cancer (e.g., a prostate cancer). In any method of diagnosis exist false positives and false negatives. Any one method of diagnosis does not provide 100% accuracy.

In another aspect, the present invention provides methods of providing a prognosis for a cancer in a subject. As used herein, the term “providing a prognosis” refers to providing a prediction of the probable course and outcome of a cancer such as prostate cancer, including prediction of metastasis, disease free survival, overall survival, etc. The methods can also be used to devise a suitable therapy for cancer treatment, e.g., by indicating whether or not the cancer is still at an early stage or if the cancer had advanced to a stage where aggressive therapy would be ineffective.

In general, the methods of diagnosing or providing a prognosis for a cancer comprise the steps of analyzing a tissue sample from the subject for at least one marker that is a downstream target of N-cadherin (e.g., at least one marker listed in Table 1 or Table 2 or Table 3); and determining whether or not the expression of at least one marker is altered (i.e., overexpressed or underexpressed) as compared to a control tissue sample; thereby providing a diagnosis for the cancer or providing a prognosis for the cancer. Diagnosis or prognosis involves determining the level of expression of an mRNA or protein of at least one marker of interest in a subject and then comparing that level of expression to a baseline or range. Typically, the baseline value is representative of an mRNA or protein of the marker of interest in a healthy person not suffering from cancer, as measured using a tissue sample (e.g., a tissue from a biopsy) or other biological sample such serum or blood. Variation of levels of expression of the mRNA or protein of the marker of interest in the subject from the baseline range (either up or down) indicates that the subject has a cancer or is at risk of developing a cancer.

In some embodiments, the cancer is an N-cadherin-overexpresing cancer. In some embodiments, the cancer is a urogenital cancer. In some embodiments, the cancer is prostate cancer. The cancer may be a primary cancer or a metastatic cancer.

In some embodiments, the at least one marker of interest that is a downstream target of N-cadherin is selected from the markers listed in Table 1 or Table 2 or Table 3 or Table 4. In some embodiments, the at least one marker of interest that is a downstream target of N-cadherin phorbol-12-myristate-13-acetate-induced protein 1 (PMAIP1), centrosomal protein 170 kDa (CEP170), gap junction protein gamma 1 (GJC1), zinc finger protein 281 (ZNF281), zinc finger protein 22 (ZNF22), matrix-remodelling associated 7 (MXRA7), NudE nuclear distribution gene E homolog 1 (NDE1), v-ets erythroblastosis virus E26 oncogene homolog 1 (ETS), homeobox B7 (HOXB7), ubiquitin-conjugating enzyme E2 variant 1 (UBE2V1), RecQ protein-like (RECQL), schwannomin interacting protein 1 (SCHIP1), RNA (guanine-7-) methyltransferase (RNMT), dedicator of cytokinesis 4 (DOCK4), adaptor-related protein complex 1 sigma 2 subunit (AP1S2), ankyrin repeat domain 28 (ANKRD28), acyl-CoA thioesterase 9 (ACOT9), A-kinase anchor protein 12 (AKAP12), MHC class I polypeptide-related sequence B (MICB), protein kinase D3 (PRKD3), deafness autosomal dominant 5 (DFNA5), fucosyltransferase 8 (FUT8), schlafen family member 11 (SLFN11), pleckstrin homology-like domain family A member 1 (PHLDA1), solute carrier family 43 member 3 (SLC43A3), insulin-like growth factor 2 mRNA binding protein 2 (IGF2BP2), solute carrier family 16 member 14 (SLC16A14), contractin associated protein 1 (CNTNAP1), chromosome 6 open reading frame 150 (C60RF150), X (inactive)-specific transcript (XIST), or fatty acyl coA reductase 2 (FAR2).

Extracellular and membrane-associated molecules are particularly attractive targets for diagnostic, prognostic, and therapeutic purposes. Thus, in some embodiments, the at least one marker of interest that is a downstream target of N-cadherin is selected from the markers listed in Table 1 or Table 2 or Table 3 or Table 4, wherein the at least one marker is expressed extracellularly or on the surface of a cell.

In some embodiments, the tissue is prostate tissue. In some embodiments, the tissue sample is a metastatic tissue sample. In some embodiments, the tissue sample is a tissue from a biopsy, such as from a urogenital tissue (e.g., prostate tissue). In some embodiments, the tissue sample is serum.

In some embodiments, a positive diagnosis for a cancer is indicated when a higher level of mRNA or protein of the at least one marker of interest is detected in a test tissue sample in comparison to a control tissue sample from an individual known not to have cancer, for example, 10%, 20%, 30%, 40%, 50%, 60%, 70%, 80%, 90%, 100%, 2-fold, 3-fold, 4-fold higher or more.

The detection methods for diagnosing a subject or providing a prognosis to a subject can be carried out, for example, using standard nucleic acid and/or polypeptide detection techniques known in the art. Detection can be accomplished by labeling a nucleic acid probe or a primary antibody or secondary antibody with, for example, a radioactive isotope, a fluorescent label, an enzyme or any other detectable label known in the art.

Antibody reagents can be used in assays to detect protein expression levels for the at least one marker of interest in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody to the protein of interest can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), in the physical form of sticks, sponges, papers, wells, and the like. An assay strip can be prepared by coating the antibody or a plurality of antibodies in an array on a solid support. This strip can then be dipped into the test sample and processed quickly through washes and detection steps to generate a measurable signal, such as a colored spot.

Alternatively, nucleic acid binding molecules such as probes, oligonucleotides, oligonucleotide arrays, and primers can be used in assays to detect differential RNA expression of the marker of interest in subject samples, e.g., RT-PCR. In one embodiment, RT-PCR is used according to standard methods known in the art. In another embodiment, PCR assays such as Taqman® assays available from, e.g., Applied Biosystems, can be used to detect nucleic acids and variants thereof. In other embodiments, qPCR and nucleic acid microarrays can be used to detect nucleic acids. Reagents that bind to selected markers of interest can be prepared according to methods known to those of skill in the art or purchased commercially.

Analysis of nucleic acids can be achieved using routine techniques such as Southern analysis, reverse-transcriptase polymerase chain reaction (RT-PCR), or any other methods based on hybridization to a nucleic acid sequence that is complementary to a portion of the marker coding sequence (e.g., slot blot hybridization) are also within the scope of the present invention. Applicable PCR amplification techniques are described in, e.g., Ausubel et al. and Innis et al., supra. General nucleic acid hybridization methods are described in Anderson, “Nucleic Acid Hybridization,” BIOS Scientific Publishers, 1999. Amplification or hybridization of a plurality of nucleic acid sequences (e.g., genomic DNA, mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression From Experiments to Data Analysis and Modeling,” Cambridge University Press, 2002.

Analysis of nucleic acid markers can also be performed using techniques known in the art including, without limitation, microarrays, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nat. Biotechnol., 16:381-384 (1998)), and sequencing by hybridization. Chee et al., Science, 274:610-614 (1996); Drmanac et al., Science, 260:1649-1652 (1993); Drmanac et al., Nat. Biotechnol., 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Other methods for detecting nucleic acid variants include, e.g., the INVADER® assay from Third Wave Technologies, Inc., restriction fragment length polymorphism (RFLP) analysis, allele-specific oligonucleotide hybridization, a heteroduplex mobility assay, single strand conformational polymorphism (SSCP) analysis, single-nucleotide primer extension (SNUPE) and pyrosequencing.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Useful physical formats comprise surfaces having a plurality of discrete, addressable locations for the detection of a plurality of different markers. Such formats include microarrays and certain capillary devices. See, e.g., Ng et al., J. Cell Mol. Med., 6:329-340 (2002); U.S. Pat. No. 6,019,944. In these embodiments, each discrete surface location may comprise antibodies to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection. Other useful physical formats include sticks, wells, sponges, and the like.

Analysis can be carried out in a variety of physical formats. For example, the use of microtiter plates or automation could be used to facilitate the processing of large numbers of test samples. Alternatively, single sample formats could be developed to facilitate diagnosis or prognosis in a timely fashion.

Alternatively, the antibodies or nucleic acid probes of the invention can be applied to subject samples immobilized on microscope slides. The resulting antibody staining or in situ hybridization pattern can be visualized using any one of a variety of light or fluorescent microscopic methods known in the art.

Analysis of the protein or nucleic acid can also be achieved, for example, by high pressure liquid chromatography (HPLC), alone or in combination with mass spectrometry (e.g., MALDI/MS, MALDI-TOF/MS, tandem MS, etc.).

### IV. Compositions, Kits, and Integrated Systems

The invention provides compositions, kits and integrated systems for practicing the assays described herein using antibodies specific for the proteins or nucleic acids specific for the markers of the invention.

Kits for carrying out the diagnostic and prognostic assays for determining the amount of protein of the marker that is a downstream target of N-cadherin typically include a detection agent that comprises an antibody (a polyclonal or monoclonal antibody, or an antiserum) that specifically binds to the target protein. Optionally, a detectable label is conjugated to the detection agent for indicating the presence of the agent and therefore the marker protein. In some cases, the kits may include multiple antibodies for detection purposes. For examples, a primary antibody and a secondary antibody may be included in the kits, with the primary antibody having a binding specificity for the marker protein, and the secondary antibody having a binding specificity for the primary antibody and having a detectable label or moiety.

Kits for carrying out diagnostic and prognostic assays for determining the amount of nucleic acid of the marker that is a downstream target of N-cadherin typically include at least one oligonucleotide useful for specific hybridization with the marker coding sequence or complementary sequence. Optionally, this oligonucleotide is labeled with a detectable moiety. In some cases, the kits may include at least two oligonucleotide primers that can be used in the amplification of the marker nucleic acid by PCR, e.g., by RT-qPCR.

Optionally, the kits also provide instruction manuals to guide users in analyzing test samples and assessing the presence or severity of a cancer (e.g. prostate cancer) in a test subject.

### V. Methods to Identify Compounds

A variety of methods may be used to identify compounds that prevent or treat a cancer expressing N-cadherin or exhibiting EMT. Typically, an assay that provides a readily measured parameter is adapted to be performed in the wells of multi-well plates in order to facilitate the screening of members of a library of test compounds as described herein. Thus, in one embodiment, an appropriate number of cells can be plated into the cells of a multi-well plate, and the effect of a test compound on the expression of a marker that is a downstream target of N-cadherin can be determined.

The compounds to be tested can be any small chemical compound, or a macromolecule, such as a protein, sugar, nucleic acid or lipid. Essentially any chemical compound can be used as a test compound in this aspect of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, high throughput screening methods are used which involve providing a combinatorial chemical or peptide library containing a large number of potential therapeutic compounds. Such “combinatorial chemical libraries” or “ligand libraries” are then screened in one or more assays, as described herein, to identify those library members (particular chemical species or subclasses) that display a desired characteristic activity. In this instance, such compounds are screened for their ability to reduce or increase the expression of one or more markers that is a downstream target of N-cadherin.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries are well known to those of skill in the art. Such combinatorial chemical libraries include, but are not limited to, peptide libraries (see, e.g., U.S. Pat. No. 5,010,175, Furka, Int. J. Pept. Prot. Res., 37:487-493 (1991) and Houghton et al., Nature, 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., PNAS USA, 90:6909-6913 (1993)), vinylogous polypeptides (Hagihara et al., J. Amer. Chem. Soc., 114:6568 (1992)), nonpeptidal peptidomimetics with glucose scaffolding (Hirschmann et al., J. Amer. Chem. Soc., 114:9217-9218 (1992)), analogous organic syntheses of small compound libraries (Chen et al., J. Amer. Chem. Soc., 116:2661 (1994)), oligocarbamates (Cho et al., Science, 261:1303 (1993)), and/or peptidyl phosphonates (Campbell et al., J. Org. Chem., 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100-about 1500 different compounds. It is possible to assay many plates per day; assay screens for up to about 6,000, 20,000, 50,000, or 100,000 or more different compounds is possible using the integrated systems of the invention.

### VI. Therapeutic Methods

In another aspect, the present invention provides methods of treating a cancer expressing N-cadherin or exhibiting EMT by targeting at least one marker that is a downstream target of N-cadherin (e.g., at least one marker listed in Table 1 or Table 2). The terms “treating” or “treatment” include:
(1) preventing the disease, i.e., causing the clinical symptoms of the disease not to develop in a mammal that may be exposed to the organism but does not yet experience or display symptoms of the disease,
(2) inhibiting the disease, i.e., arresting or reducing the development of the disease or its clinical symptoms. This includes reducing the extent of the detachment observed or the numbers of subjects or risk of a subject having a detachment.
(3) relieving the disease, i.e., causing regression of the disease or its clinical symptoms.

In some embodiments, the method comprises administering to a subject having a cancer expressing N-cadherin or exhibiting EMT a therapeutically effective amount of an antibody that specifically binds to the marker that is a downstream target of N-cadherin. In some embodiments, the method comprises administering to a subject having a cancer expressing N-cadherin or exhibiting EMT a therapeutically effective amount of an inhibitory oligonucleotide (e.g., siRNA, antisense nucleic acid, aptamer, or ribozyme) that inhibits the expression and/or activity of the marker that is a downstream target of N-cadherin. In some embodiments, the method comprises administering to a subject having a cancer expressing N-cadherin or exhibiting EMT a therapeutically effective amount of an inhibitory small molecule that inhibits the expression and/or activity of the marker that is a downstream target of N-cadherin.

By “therapeutically effective dose or amount” herein is meant a dose that produces effects for which it is administered. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20^{th} Edition, Gennar, Editor (2003); and Pickar, Dosage Calculations (1999)). The antibodies, inhibitory nucleic acids, and/or small molecules as described herein for use in the present invention may be administered by any route of administration (e.g., intravenous, topical, intraperitoneal, parenteral, oral, intravaginal, rectal, ocular, intravitreal and intraocular). They may be administered as a bolus or by continuous infusion over a period of time, by intramuscular, intraperitoneal, subcutaneous, oral, topical, or inhalation routes. Intravenous or subcutaneous administration of the antibody is preferred. The administration may be local or systemic. They may be administered to a subject who has been diagnosed with the subject disease, a history of the disease, or is at risk of the disease.

In some embodiments, antibodies can be used to inhibit the function of the markers that are downstream targets of N-cadherin. Said antibodies may be used systemically to treat cancer (e.g., prostate cancer) alone or when conjugated with an effector moiety. In some embodiments, the effector moiety is a therapeutic moiety. Examples of effector moieties include, but are not limited to, an anti-tumor drug, a toxin, a radioactive agent, a cytokine, a second antibody, or an enzyme. In some embodiments, the antibody that targets the marker that is a downstream target of N-cadherin is linked to an enzyme that converts a prodrug into a cytotoxic agent.

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

In some embodiments, inhibitory nucleic acids can be used to inhibit the function of the markers that are downstream targets of N-cadherin. A wide variety of nucleic acids, such as antisense nucleic acids, siRNAs or ribozymes, may be used to inhibit the function of the markers of this invention. Ribozymes that cleave mRNA at site-specific recognition sequences can be used to destroy target mRNAs, particularly through the use of hammerhead ribozymes. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

With regard to antisense, siRNA or ribozyme oligonucleotides, phosphorothioate oligonucleotides can be used. Modifications of the phosphodiester linkage as well as of the heterocycle or the sugar may provide an increase in efficiency. Phophorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-0-propyl and 2′-methoxyethoxy ribose modifications stabilize oligonucleotides to nucleases in cell culture and in vivo.

Inhibitory oligonucleotides can be delivered by direct transfection or transfection and expression via an expression vector. Appropriate expression vectors include mammalian expression vectors and viral vectors, into which has been cloned an inhibitory oligonucleotide with the appropriate regulatory sequences including a promoter to result in expression of the antisense RNA in a host cell. Suitable promoters can be constitutive or development-specific promoters. Transfection delivery can be achieved by liposomal transfection reagents, known in the art (e.g., Xtreme transfection reagent, Roche, Alameda, Calif.; Lipofectamine formulations, Invitrogen, Carlsbad, Calif.). Delivery mediated by cationic liposomes, by retroviral vectors and direct delivery are efficient. Another possible delivery mode is targeting using antibody to cell surface markers for the target cells (e.g., cancer cells).

For transfection, a composition comprising one or more nucleic acid molecules (within or without vectors) can comprise a delivery vehicle, including liposomes, for administration to a subject, carriers and diluents and their salts, and/or can be present in pharmaceutically acceptable formulations. Methods for the delivery of nucleic acid molecules are described, for example, in Gilmore, et al., Curr Drug Delivery (2006) 3:147-5 and Patil, et al., AAPS Journal (2005) 7:E61-E77, each of which are incorporated herein by reference. Delivery of siRNA molecules is also described in several U.S. Patent Publications, including for example, 2006/0019912; 2006/0014289; 2005/0239687; 2005/0222064; and 2004/0204377, the disclosures of each of which are hereby incorporated herein by reference. Nucleic acid molecules can be administered to cells by a variety of methods known to those of skill in the art, including, but not restricted to, encapsulation in liposomes, by iontophoresis, by electroporation, or by incorporation into other vehicles, including biodegradable polymers, hydrogels, cyclodextrins (see, for example Gonzalez et al., 1999, Bioconjugate Chem., 10, 1068-1074; Wang et al., International PCT publication Nos. WO 03/47518 and WO 03/46185), poly(lactic-co-glycolic)acid (PLGA) and PLCA microspheres (see for example U.S. Pat. No. 6,447,796 and US Patent Application Publication No. 2002/130430), biodegradable nanocapsules, and bioadhesive microspheres, or by proteinaceous vectors (O'Hare and Normand, International PCT Publication No. WO 00/53722). In another embodiment, the nucleic acid molecules of the invention can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

Examples of liposomal transfection reagents of use with this invention include, for example: CellFectin, 1:1.5 (M/M) liposome formulation of the cationic lipid N,NI,NII,NIII-tetramethyl-N,NI,NII,NIII-tetrapalmit-y-spermine and dioleoyl phosphatidylethanolamine (DOPE) (GIBCO BRL); Cytofectin GSV, 2:1 (M/M) liposome formulation of a cationic lipid and DOPE (Glen Research); DOTAP (N-[1-(2,3-dioleoyloxy)-N,N,N-tri-methyl-ammoniummethylsulfate) (Boehringer Manheim); Lipofectamine, 3:1 (M/M) liposome formulation of the polycationic lipid DOSPA and the neutral lipid DOPE (GIBCO BRL); and (5) siPORT (Ambion); HiPerfect (Qiagen); X-treme GENE (Roche); RNAicarrier (Epoch Biolabs) and TransPass (New England Biolabs).

In some embodiments, antisense, siRNA, or ribozyme sequences are delivered into cells (e.g., cancer cells) via a mammalian expression vector. For example, mammalian expression vectors suitable for siRNA expression are commercially available, for example, from Ambion (e.g., pSilencer vectors), Austin, Tex.; Promega (e.g., GeneClip, siSTRIKE, SiLentGene), Madison, Wis.; Invitrogen, Carlsbad, Calif.; InvivoGen, San Diego, Calif.; and Imgenex, San Diego, Calif.

In some embodiments, antisense, siRNA, or ribozyme sequences are delivered into cells (e.g., cancer cells) via a viral expression vector. Viral vectors suitable for delivering such molecules to cells include adenoviral vectors, adeno-associated vectors, and retroviral vectors (including lentiviral vectors). For example, viral vectors developed for delivering and expressing siRNA oligonucleotides are commercially available from, for example, GeneDetect, Bradenton, Fla.; Ambion, Austin, Tex.; Invitrogen, Carlsbad, Calif.; Open BioSystems, Huntsville, Ala.; and Imgenex, San Diego, Calif.

### EXAMPLES

The following examples are offered to illustrate, but not to limit the claimed invention.

### Example 1

A set of genes are described which were found to be upregulated or down-regulated in prostate cancer cell lines that were engineered to express varying levels of N-cadherin. The gene set was evaluated in multiple ways, including comparison to public datasets of genes associated with prostate cancer metastasis. Genes of interest were also selected based on putative function and suitability for therapeutic targetings, such as kinases, cell surface proteins, and transcription factors. Genes that met multiple criteria were then evaluated in the prostate cancer cell lines to confirm their expression, and in varying grades of primary prostate cancer.

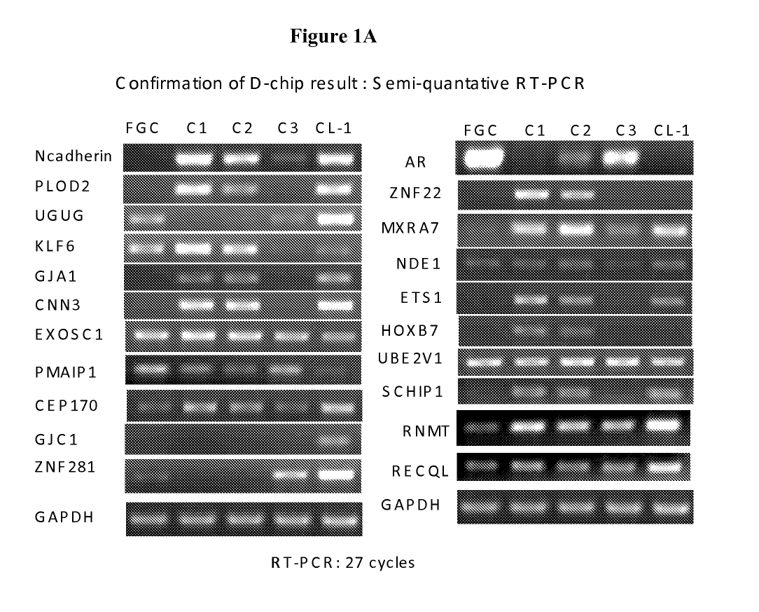

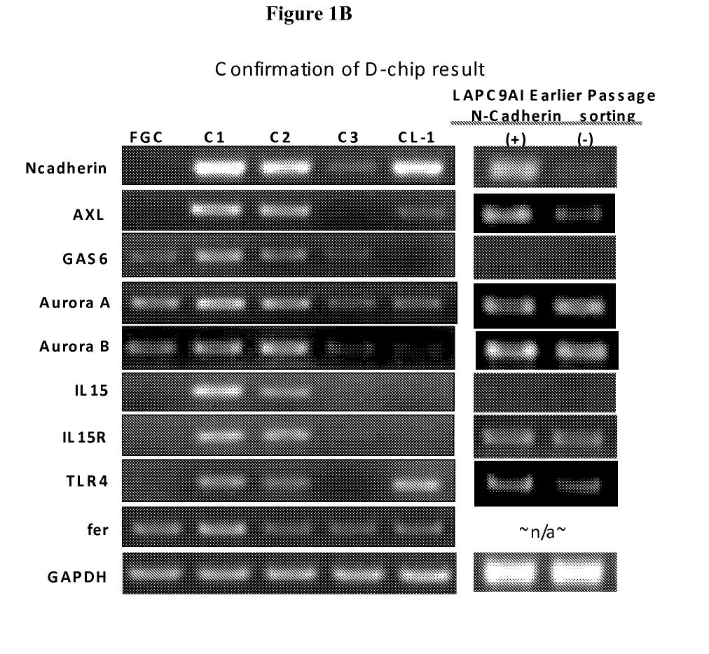

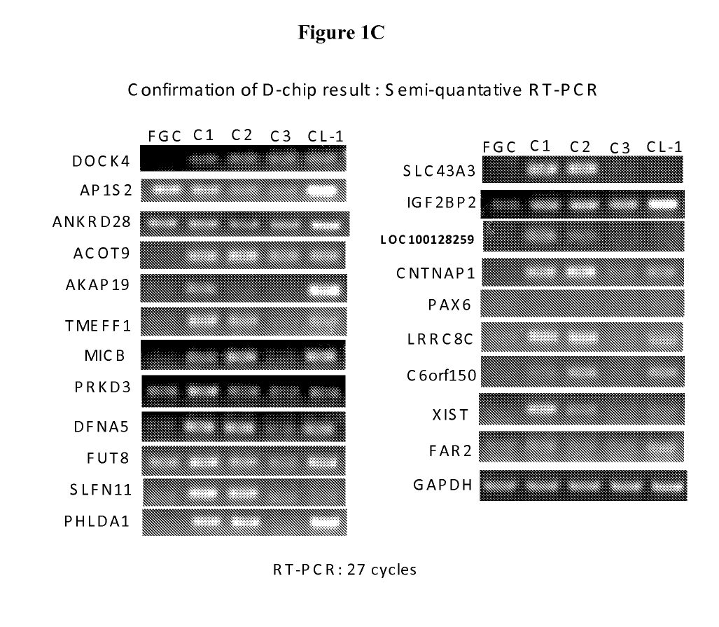

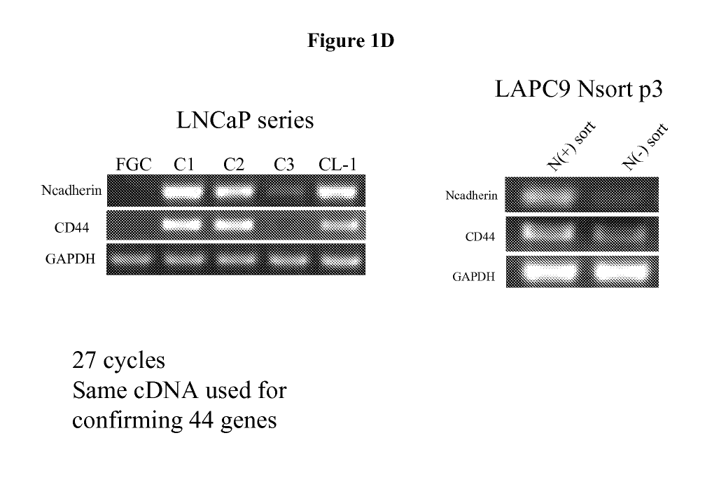

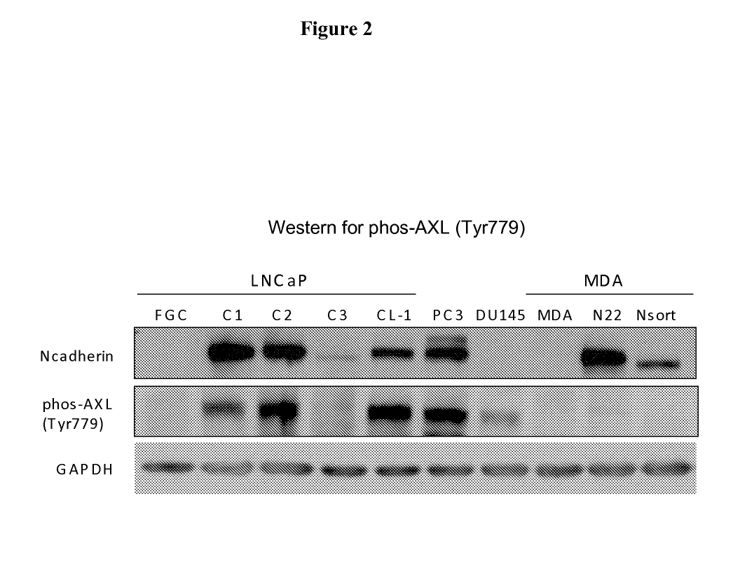

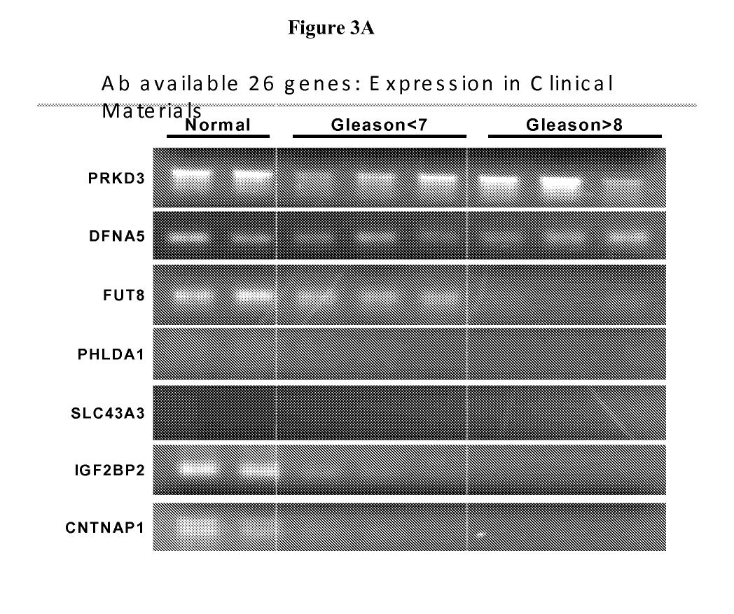

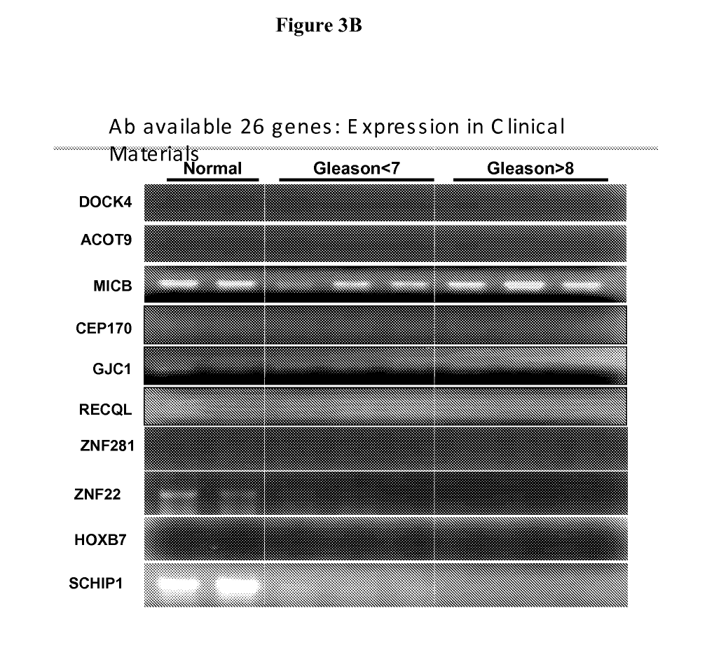

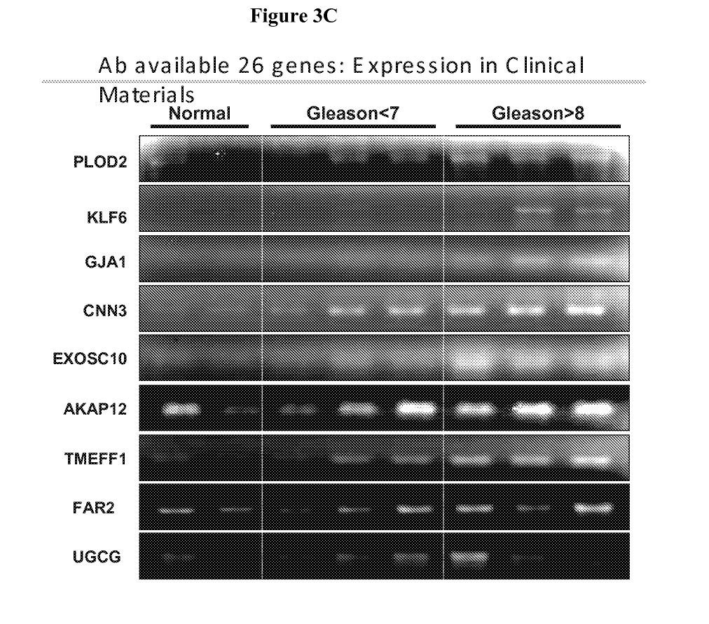

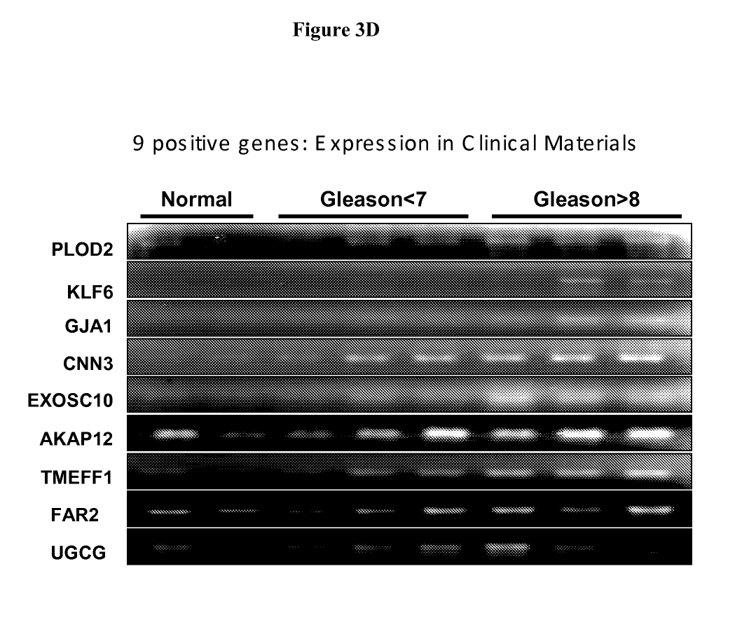

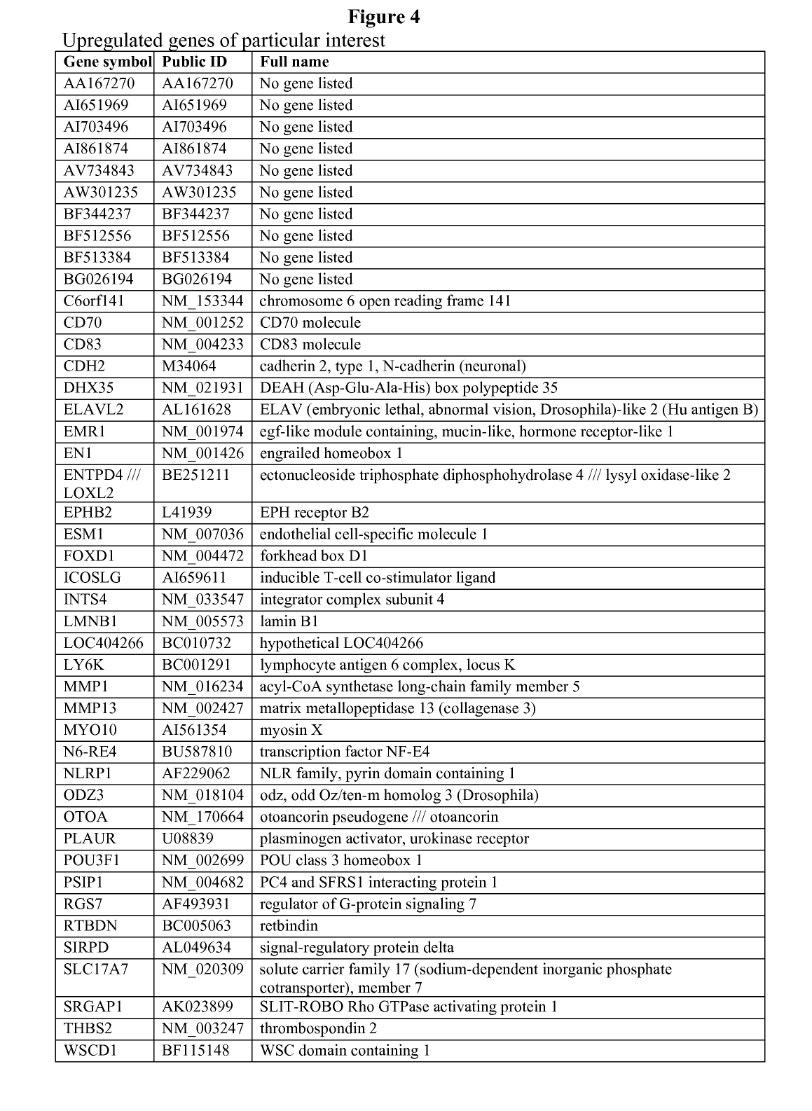

RNA was generated from LNCaP, LNCaP C1, LNCaP C2, and LNCaP C3 lines (LNCaP cell lines transduced with varying levels of N-cadherin; LNCaP C1 is a high expressing N-cadherin line, LNCaP C2 is an intermediate expressing N-cadherin line, and LNCaP C3 is a low expressing N-cadherin line). We also compared gene expression in the MDA-Pca2b cell line transduced with N-cadherin (“MDA-N”). Gene expression was compared using Affymetrix HG-133 Plus 2.0 Arrays, which contains more than 54,000 probe sets used to analyze the expression of more than 47,000 transcripts and variants, including at least 38,500 well characterized human genes. Full chip service including hybridization, scanning, and data extraction was done by the UCLA DNA Microarray Core Facility. Analysis was performed using “R” software. Comparison was done between LNCaP C1 vs. C2 and C3 (looking at genes upregulated in C1), and MDA vs. MDA-N cells. Expression was based on statistically significant p and q values. In addition, the genes of interest were also statistically significant against 7 prostate cancer published arrays. 60 upregulated genes of interest were selected. Confirmation of microarray data was performed on cell lines and clinical metastatic samples using RT-PCR (FIG. 1) and Western blot (FIGS. 2-3) analysis to confirm 49 genes as downstream of N-cadherin and associated with EMT (Table 1).

TABLE 1
Markers upregulated in N-cadherin-expressing prostate cancer tissues

| Accession ID | Gene Name and Abbreviation |
|---|---|
| NM_000935.1 | procollagen-lysine, 2-oxoglutarate 5-dioxygenase 2 (PLOD2) |
| NM_003358.1 | UDP-glucose ceramide glucosyltransferase (UGCG) |
| BU683415 | DNA-binding protein CPBP (CPBP; also KLF6) |
| NM_000165.2 | gap junction protein, alpha 1, 43 kDa (GJA1) |
| AI807004 | calponin 3, acidic (CNN3) |
| NM_002685.1 | exosome component 10 (EXOSC1) |
| AI857639 | phorbol-12-myristate-13-acetate-induced protein 1 (PMAIP1) |
| NM_014812.1 | centrosomal protein 170 kDa (KARP-binding protein) (CEP170) |
| AA430014 | gap junction protein, gamma 1, 45 kDa (GJC1) |
| AU150752 | zinc finger protein 281 (ZNF281) |
| AA744771 | zinc finger protein 22 (ZNF22; also KOX 15) |
| BF968134 | matrix-remodelling associated 7 (MXRA7) |
| AI857685 | NudE nuclear distribution gene E homolog 1 (A. nidulans), mRNA (cDNA |
| | clone MGC: 33664 IMAGE: 4828494) (NDE1) |
| BE218980 | v-ets erythroblastosis virus E26 oncogene homolog 1 (avian) (ETS1) |
| S49765.1 | homeobox B7 (HOXB7) |
| BG164064 | TMEM189-UBE2V1 readthrough transcript /// ubiquitin-conjugating enzyme |
| | E2 variant 1 (UBE2V1) |
| AI962943 | RecQ protein-like (DNA helicase Q1-like) (RECQL) |
| NM_014575.1 | schwannomin interacting protein 1 (SCHIP1) |
| NM_003799.1 | RNA (guanine-7-) methyltransferase (RNMT) |
| NM_014705.1 | dedicator of cytokinesis 4 (DOCK4) |
| AA551090 | adaptor-related protein complex 1, sigma 2 subunit (AP1S2) |
| AI081194 | ankyrin repeat domain 28 (ANKRD28) |
| AF241787.1 | acyl-CoA thioesterase 9 (ACOT9) |
| NM_005100.3 | A kinase anchor protein 12, isoform 1 (AKAP12) |
| BF439316 | transmembrane protein with EGF-like and two follistatin-like domains 1 |
| | (TMEFF1) |
| NM_005931.1 | MHC class I polypeptide-related sequence B (MICB) |
| Z25429.1 | protein kinase D3 (PRKD3) |
| NM_004403.1 | deafness, autosomal dominant 5 (DFNA5) |
| NM_004480.1 | fucosyltransferase 8 (alpha (1,6) fucosyltransferase) (FUT8) |
| AW003459 | schlafen family member 11 (SLFN11) |
| AI795908 | pleckstrin homology-like domain, family A, member 1 (PHLDA1) |
| AI630178 | solute carrier family 43, member 3 (SLC43A3) |
| NM_006548.1 | insulin-like growth factor 2 mRNA binding protein 2 (IGF2BP2) |
| AW196959 | hypothetical protein LOC100128259 /// similar to solute carrier family 16 |
| | (monocarboxylic acid transporters), member 14 (LOC1001128259) |
| NM_003632.1 | contactin associated protein 1 (CNTNAP1) |
| NM_000280.1 | paired box 6 (PAX6) |
| BE877357 | leucine rich repeat containing 8 family, member C (LRRC8C) |
| AK097148.1 | chromosome 6 open reading frame 150 (C6ORF150) |
| AV699347 | X (inactive)-specific transcript (non-protein coding) (XIST) |
| H16791 | Fatty acyl CoA reductase 2, mRNA (cDNA clone MGC: 22328 |
| | IMAGE: 4732586) (FAR2) |

### Example 2

A set of genes are described which were found to be upregulated or down-regulated in prostate cancer cell lines that were engineered to express varying levels of N-cadherin. The gene set was evaluated in multiple ways, including comparison to public datasets of genes associated with prostate cancer metastasis. The list of genes was generated based on a 1.5× fold difference in expression between localized and metastatic sets. Genes of interest were also selected based on putative function and suitability for therapeutic targetings, such as kinases, cell surface proteins, and transcription factors. Genes that met multiple criteria were then evaluated in the prostate cancer cell lines to confirm their expression, and in varying grades of primary prostate cancer.

RNA was generated from LNCaP, LNCaP C1, LNCaP C2, and LNCaP C3 lines (LNCaP cell lines transduced with varying levels of N-cadherin). We also compared gene expression in the MDA-Pca2b cell line transduced with N-cadherin. Gene expression was compared using Affymetrix HG-133 Plus 2.0 Arrays, which contains more than 54,000 probe sets used to analyze the expression of more than 47,000 transcripts and variants, including at least 38,500 well characterized human genes. Full chip service including hybridization, scanning, and data extraction was done by the UCLA DNA Microarray Core Facility. Analysis was performed using “R” software. Comparison was done between LNCaP C1 vs. C2 and C3 (looking at genes upregulated in C1), MDA vs. MDA-N cells, and public database Varambally. Expression was based on statistically significant p and q values. In addition, the genes of interest were also statistically significant against 7 prostate cancer published arrays. 722 upregulated genes of interest were selected. Confirmation of microarray data was performed on cell lines and clinical metastatic samples to confirm 512 genes as downstream of N-cadherin and associated with EMT (Table 2).

TABLE 2
Markers upregulated in N-cadherin-expressing prostate cancer tissues

| Probe Set ID | |
|---|---|
| (Affymetrix) | Gene Symbol |
| 223381_at | NUF2 |
| 201291_s_at | TOP2A |
| 212094_at | PEG10 |
| 202410_x_at | IGF2 |
| 225834_at | AL135396 |
| 210103_s_at | FOXA2 |
| 202954_at | UBE2C |
| 208079_s_at | AURKA |
| 209773_s_at | RRM2 |
| 215509_s_at | AL137654 |
| 210052_s_at | TPX2 |
| 209408_at | KIF2C |
| 235709_at | GAS2L3 |
| 205081_at | CRIP1 |
| 202870_s_at | CDC20 |
| 204825_at | MELK |
| 219956_at | GALNT6 |
| 243840_at | BF691634 |
| 205046_at | CENPE |
| 205366_s_at | HOXB6 |
| 203438_at | STC2 |
| 206364_at | KIF14 |
| 229490_s_at | AW271106 |
| 219170_at | FSD1 |
| 218009_s_at | PRC1 |
| 228273_at | BG165011 |
| 232238_at | ASPM |
| 228033_at | E2F7 |
| 207828_s_at | CENPF |
| 205646_s_at | PAX6 |
| 203764_at | DLGAP5 |
| 219911_s_at | SLCO4A1 |
| 218355_at | KIF4A |
| 205402_x_at | PRSS2 |
| 216973_s_at | HOXB7 |
| 226299_at | PKN3 |
| 215116_s_at | DNM1 |
| 207165_at | HMMR |
| 238756_at | AI860012 |
| 230935_at | AI861874 |
| 221258_s_at | KIF18A |
| 203069_at | SV2A |
| 228323_at | CASC5 |
| 219493_at | SHCBP1 |
| 202503_s_at | KIAA0101 |
| 201664_at | SMC4 |
| 231938_at | SGOL1 |
| 218662_s_at | NCAPG |
| 218726_at | HJURP |
| 217640_x_at | C18orf24 |
| 201853_s_at | CDC25B |
| 214804_at | BF793446 |
| 1553713_a_at | RHEBL1 |
| 204936_at | MAP4K2 |
| 214639_s_at | HOXA1 |
| 206157_at | PTX3 |
| 226980_at | DEPDC1B |
| 222848_at | CENPK |
| 229610_at | CKAP2L |
| 208998_at | UCP2 |
| 210220_at | FZD2 |
| 221677_s_at | DONSON |
| 204641_at | NEK2 |
| 204584_at | L1CAM |
| 212909_at | LYPD1 |
| 212801_at | CIT |
| 219588_s_at | NCAPG2 |
| 204822_at | TTK |
| 218542_at | CEP55 |
| 226281_at | DNER |
| 208725_at | LOC100130797 |
| 208510_s_at | PPARG |
| 232105_at | AU148391 |
| 225612_s_at | B3GNT5 |
| 203432_at | AW272611 |
| 242890_at | AI650364 |
| 222608_s_at | ANLN |
| 206247_at | MICB |
| 205453_at | HOXB2 |
| 213226_at | CCNA2 |
| 205522_at | HOXD4 |
| 209446_s_at | C7orf44 |
| 204413_at | TRAF2 |
| 205167_s_at | CDC25C |
| 202651_at | LPGAT1 |
| 230664_at | H09657 |
| 235609_at | BF056791 |
| 208962_s_at | FADS1 |
| 204444_at | KIF11 |
| 227212_s_at | PHF19 |
| 213358_at | KIAA0802 |
| 208808_s_at | HMGB2 |
| 204285_s_at | PMAIP1 |
| 228564_at | LOC375295 |
| 1552712_a_at | NMNAT2 |
| 227405_s_at | FZD8 |
| 201897_s_at | CKS1B |
| 204886_at | PLK4 |
| 213378_s_at | DDX12 |
| 227249_at | AI857685 |
| 203980_at | FABP4 |
| 228904_at | HOXB3 |
| 214710_s_at | CCNB1 |
| 236513_at | AW770245 |
| 229485_x_at | SHISA3 |
| 205260_s_at | ACYP1 |
| 202620_s_at | PLOD2 |
| 221922_at | GPSM2 |
| 203805_s_at | FANCA |
| 238587_at | UBASH3B |
| 222958_s_at | DEPDC1 |
| 224774_s_at | NAV1 |
| 230493_at | SHISA2 |
| 223484_at | C15orf48 |
| 208978_at | CRIP2 |
| 219863_at | HERC5 |
| 225898_at | WDR54 |
| 223542_at | ANKRD32 |
| 1556346_at | AJ227860 |
| 206508_at | CD70 |
| 204411_at | KIF21B |
| 204729_s_at | STX1A |
| 1560527_at | BU587810 |
| 221505_at | ANP32E |
| 222557_at | RTEL1 |
| 209464_at | AURKB |
| 210847_x_at | TNFRSF25 |
| 205899_at | CCNA1 |
| 227350_at | AI889959 |
| 221059_s_at | CHST6 |
| 227349_at | AI807356 |
| 1558871_at | BC016361 |
| 243502_at | BF035598 |
| 224428_s_at | CDCA7 |
| 214604_at | HOXD11 |
| 239253_at | AI926924 |
| 209435_s_at | BC000265 |
| 229400_at | HOXD10 |
| 238537_at | AA330389 |
| 219888_at | SPAG4 |
| 220658_s_at | ARNTL2 |
| 211935_at | ARL6IP1 |
| 221969_at | BF510692 |
| 1555907_at | LOC100130776 |
| 1568813_at | BC009525 |
| 208767_s_at | LAPTM4B |
| 241541_at | MIB2 |
| 214772_at | C11orf41 |
| 227072_at | RTTN |
| 201564_s_at | FSCN1 |
| 207541_s_at | EXOSC10 |
| 224724_at | SULF2 |
| 225681_at | CTHRC1 |
| 224944_at | AL566034 |
| 218768_at | NUP107 |
| 209421_at | MSH2 |
| 224583_at | COTL1 |
| 205339_at | STIL |
| 219523_s_at | NM_018104 |
| 204146_at | BE966146 |
| 211208_s_at | CASK |
| 221685_s_at | CCDC99 |
| 227146_at | QSOX2 |
| 202733_at | P4HA2 |
| 1558750_a_at | BG109249 |
| 226063_at | VAV2 |
| 235205_at | LOC100128259 |
| 201578_at | PODXL |
| 200916_at | TAGLN2 |
| 1557852_at | AW418842 |
| 205122_at | TMEFF1 |
| 206343_s_at | NRG1 |
| 228737_at | TOX2 |
| 218611_at | IER5 |
| 230165_at | SGOL2 |
| 227841_at | CEMP1 |
| 212552_at | BE617588 |
| 206805_at | SEMA3A |
| 226265_at | QSER1 |
| 224598_at | MGAT4B |
| 223700_at | MND1 |
| 211824_x_at | NLRP1 |
| 201636_at | FXR1 |
| 218781_at | SMC6 |
| 238445_x_at | MGAT5B |
| 206550_s_at | NUP155 |
| 220223_at | ATAD5 |
| 220840_s_at | C1orf112 |
| 209891_at | SPC25 |
| 209049_s_at | BC001004 |
| 210933_s_at | BC004908 |
| 224320_s_at | MCM8 |
| 229097_at | DIAPH3 |
| 219937_at | TRHDE |
| 222619_at | ZNF281 |
| 210021_s_at | CCNO |
| 207113_s_at | TNF |
| 1565951_s_at | CHML |
| 200762_at | DPYSL2 |
| 211031_s_at | CLIP2 |
| 228776_at | GJC1 |
| 203262_s_at | FAM50A |
| 242005_at | BE877420 |
| 220091_at | SLC2A6 |
| 229128_s_at | AI697657 |
| 241937_s_at | AA577678 |
| 228593_at | LOC339483 |
| 230945_at | AI014551 |
| 219512_at | DSN1 |
| 205176_s_at | ITGB3BP |
| 204073_s_at | C11orf9 |
| 218875_s_at | FBXO5 |
| 213135_at | TIAM1 |
| 1552680_a_at | NM_020380 |
| 204033_at | TRIP13 |
| 222549_at | CLDN1 |
| 229700_at | BE966267 |
| 226743_at | SLFN11 |
| 200661_at | CTSA |
| 236718_at | MYO10 |
| 227530_at | AKAP12 |
| 209789_at | CORO2B |
| 231067_s_at | BF114967 |
| 207629_s_at | ARHGEF2 |
| 209627_s_at | OSBPL3 |
| 205569_at | LAMP3 |
| 210896_s_at | ASPH |
| 218088_s_at | RRAGC |
| 208736_at | ARPC3 |
| 204158_s_at | TCIRG1 |
| 203257_s_at | C11orf49 |
| 223556_at | HELLS |
| 221703_at | BRIP1 |
| 204677_at | CDH5 |
| 205296_at | SAMHD1 |
| 221485_at | B4GALT5 |
| 213065_at | CCDC131 |
| 201558_at | RAE1 |
| 1559051_s_at | C6orf150 |
| 214520_at | FOXC2 |
| 222281_s_at | AW517716 |
| 231767_at | HOXB4 |
| 224955_at | AI590088 |
| 218576_s_at | DUSP12 |
| 213532_at | AI797833 |
| 219530_at | PALB2 |
| 202656_s_at | SERTAD2 |
| 213338_at | TMEM158 |
| 227139_s_at | HPS3 |
| 202413_s_at | USP1 |
| 1554379_a_at | TP73 |
| 226552_at | IER5L |
| 205600_x_at | HOXB5 |
| 230669_at | RASA2 |
| 205515_at | PRSS12 |
| 225288_at | AI949136 |
| 229493_at | BF315468 |
| 213309_at | PLCL2 |
| 226611_s_at | PRR6 |
| 232140_at | LOC100132352 |
| 1552691_at | ARL11 |
| 235252_at | KSR1 |
| 221269_s_at | SH3BGRL3 |
| 223974_at | MGC11082 |
| 213802_at | AI810767 |
| 229796_at | SIX4 |
| 201920_at | SLC20A1 |
| 200618_at | LASP1 |
| 218802_at | CCDC109B |
| 217294_s_at | U88968 |
| 213421_x_at | PRSS3 |
| 213573_at | KPNB1 |
| 211603_s_at | U35622 |
| 207110_at | KCNJ12 |
| 200039_s_at | PSMB2 |
| 227749_at | AI703496 |
| 1556579_s_at | IGSF10 |
| 204726_at | CDH13 |
| 201678_s_at | C3orf37 |
| 226777_at | AA147933 |
| 225614_at | SAAL1 |
| 230224_at | ZCCHC18 |
| 227443_at | C9orf150 |
| 212944_at | SLC5A3 |
| 215395_x_at | U66061 |
| 239431_at | TICAM2 |
| 205548_s_at | BTG3 |
| 225468_at | PATL1 |
| 218451_at | CDCP1 |
| 230640_at | AW027431 |
| 206074_s_at | HMGA1 |
| 229067_at | SRGAP2P1 |
| 225484_at | TSGA14 |
| 225750_at | BE966748 |
| 226582_at | LOC400043 |
| 206822_s_at | L3MBTL |
| 209165_at | AATF |
| 218643_s_at | CRIPT |
| 223626_x_at | FAM14A |
| 219569_s_at | TMEM22 |
| 219502_at | NEIL3 |
| 200833_s_at | RAP1B |
| 210212_x_at | MTCP1 |
| 208178_x_at | TRIO |
| 238604_at | AA768884 |
| 206298_at | ARHGAP22 |
| 211977_at | GPR107 |
| 204044_at | QPRT |
| 223723_at | MFI2 |
| 238402_s_at | FLJ35220 |
| 220426_at | C20orf195 |
| 204872_at | TLE4 |
| 235828_at | PRELID2 |
| 227806_at | C16orf74 |
| 218991_at | HEATR6 |
| 226017_at | CMTM7 |
| 204468_s_at | TIE1 |
| 213305_s_at | PPP2R5C |
| 1554004_a_at | RGNEF |
| 228933_at | NHS |
| 202043_s_at | SMS |
| 220746_s_at | UIMC1 |
| 217733_s_at | TMSB10 |
| 226997_at | ADAMTS12 |
| 225439_at | NUDCD1 |
| 227484_at | BF508615 |
| 204475_at | MMP1 |
| 228498_at | AV687517 |
| 214051_at | MGC39900 |
| 220253_s_at | LRP12 |
| 221484_at | BF691447 |
| 201774_s_at | NCAPD2 |
| 202760_s_at | PALM2-AKAP2 |
| 226335_at | RPS6KA3 |
| 230362_at | INPP5F |
| 216222_s_at | AI561354 |
| 201939_at | PLK2 |
| 201808_s_at | ENG |
| 201266_at | TXNRD1 |
| 209122_at | ADFP |
| 202411_at | IFI27 |
| 1555962_at | B3GNT7 |
| 224116_at | BC003588 |
| 204789_at | FMNL1 |
| 217992_s_at | EFHD2 |
| 201037_at | PFKP |
| 212898_at | KIAA0406 |
| 213741_s_at | KPNA1 |
| 207624_s_at | RPGR |
| 227786_at | MED30 |
| 213090_s_at | TAF4 |
| 235530_at | AI986112 |
| 227367_at | AW976431 |
| 234932_s_at | AK026028 |
| 205969_at | AADAC |
| 218056_at | BFAR |
| 211318_s_at | U85943 |
| 51176_at | MED27 |
| 223831_x_at | ISY1 |
| 220334_at | RGS17 |
| 225234_at | CBL |
| 201995_at | EXT1 |
| 205781_at | C16orf7 |
| 201114_x_at | PSMA7 |
| 218247_s_at | MEX3C |
| 230734_x_at | AI279536 |
| 217076_s_at | HOXD3 |
| 211965_at | BE620915 |
| 231772_x_at | CENPH |
| 229667_s_at | HOXB8 |
| 226175_at | TTC9C |
| 228785_at | AA121673 |
| 218207_s_at | STMN3 |
| 213088_s_at | DNAJC9 |
| 202559_x_at | AW005776 |
| 232787_at | PRIC285 |
| 219677_at | SPSB1 |
| 203234_at | UPP1 |
| 214107_x_at | LOC729034 |
| 213035_at | ANKRD28 |
| 226614_s_at | C8orf13 |
| 202345_s_at | FABP5 |
| 230399_at | AI361034 |
| 223174_at | BTBD10 |
| 236791_at | AI820650 |
| 219229_at | SLCO3A1 |
| 205730_s_at | ABLIM3 |
| 229208_at | CEP27 |
| 209444_at | RAP1GDS1 |
| 218014_at | NUP85 |
| 202515_at | DLG1 |
| 207196_s_at | TNIP1 |
| 219007_at | NM_024647 |
| 206906_at | ICAM5 |
| 236259_at | BF433725 |
| 218600_at | LIMD2 |
| 31845_at | ELF4 |
| 201207_at | TNFAIP1 |
| 201749_at | ECE1 |
| 205076_s_at | NM_006697 |
| 217997_at | PHLDA1 |
| 220234_at | CA8 |
| 202074_s_at | OPTN |
| 201092_at | RBBP7 |
| 243613_at | MGC24039 |
| 37547_at | BBS9 |
| 205349_at | GNA15 |
| 202514_at | AW139131 |
| 1555137_a_at | FGD6 |
| 200783_s_at | STMN1 |
| 235286_at | BG533580 |
| 227828_s_at | TMEM166 |
| 223689_at | IGF2BP1 |
| 212746_s_at | AA126789 |
| 209272_at | NAB1 |
| 213977_s_at | CIZ1 |
| 225297_at | CCDC5 |
| 206581_at | BNC1 |
| 229104_s_at | GPR39 |
| 204030_s_at | SCHIP1 |
| 235044_at | H06649 |
| 218705_s_at | SNX24 |
| 224973_at | FAM46A |
| 177_at | PLD1 |
| 209000_s_at | SEPT8 |
| 221730_at | COL5A2 |
| 222590_s_at | NLK |
| 206918_s_at | CPNE1 |
| 205205_at | RELB |
| 204257_at | FADS3 |
| 219251_s_at | WDR60 |
| 212190_at | SERPINE2 |
| 201834_at | PRKAB1 |
| 202997_s_at | LOXL2 |
| 236619_at | AI922972 |
| 233085_s_at | AV734843 |
| 225008_at | AW469351 |
| 212371_at | FAM152A |
| 221666_s_at | PYCARD |
| 224796_at | DDEF1 |
| 236219_at | AI452512 |
| 224794_s_at | CERCAM |
| 200678_x_at | GRN |
| 213346_at | C13orf27 |
| 204881_s_at | UGCG |
| 241394_at | LOC284120 |
| 228843_at | AI824171 |
| 234978_at | SLC36A4 |
| 212263_at | QKI |
| 201431_s_at | DPYSL3 |
| 219268_at | ETNK2 |
| 204369_at | PIK3CA |
| 203683_s_at | VEGFB |
| 211980_at | AI922605 |
| 214853_s_at | AI091079 |
| 1553311_at | C20orf197 |
| 224701_at | PARP14 |
| 211464_x_at | CASP6 |
| 1559725_at | AL832797 |
| 203136_at | RABAC1 |
| 234950_s_at | RFWD2 |
| 207375_s_at | IL15RA |
| 244609_at | AW614107 |
| 210138_at | RGS20 |
| 216088_s_at | AL078633 |
| 200612_s_at | AP2B1 |
| 213638_at | PHACTR1 |
| 202003_s_at | ACAA2 |
| 204962_s_at | CENPA |
| 229879_at | BF059124 |
| 208433_s_at | LRP8 |
| 227975_at | GPRIN1 |
| 205206_at | KAL1 |
| 209053_s_at | BE793789 |
| 215629_s_at | DLEU2L |
| 1557051_s_at | CA448125 |
| 238949_at | RNF145 |
| 222810_s_at | RASAL2 |
| 242346_x_at | BF222929 |
| 213164_at | AI867198 |
| 244612_at | AW117181 |
| 208626_s_at | VAT1 |
| 232095_at | BG109134 |
| 224783_at | AA831661 |
| 225293_at | COL27A1 |
| 201502_s_at | NFKBIA |
| 242077_x_at | R98018 |
| 236313_at | CDKN2B |
| 205745_x_at | ADAM17 |
| 204549_at | IKBKE |
| 200960_x_at | CLTA |
| 212501_at | AL564683 |
| 203321_s_at | ADNP2 |
| 218651_s_at | LARP6 |
| 223773_s_at | C1orf79 |
| 209808_x_at | ING1 |
| 203554_x_at | PTTG1 |
| 222039_at | KIF18B |
| 218039_at | NUSAP1 |
| 207339_s_at | LTB |
| 208002_s_at | ACOT7 |
| 212983_at | HRAS |
| 213030_s_at | PLXNA2 |
| 215977_x_at | GK |
| 1555864_s_at | PDHA1 |
| 219576_at | NM_024765 |
| 201251_at | PKM2 |
| 225371_at | GLE1 |
| 1557303_at | NT5C |
| 220937_s_at | NM_014403 |
| 218902_at | NOTCH1 |

### Example 3

N-cadherin is a marker of epithelial to mesenchymal transition and is upregulated in castrate resistant prostate cancers and is associated with invasion and metastasis. The purpose of this example is to identify major downstream pathways and potential therapeutic targets for drug development regulated by N-cadherin. Prostate cancers continue to fail even the newest forms of hormone ablation therapy. N-cadherin is present in a substantial percentage of lethal prostate cancers and that some of the current treatments may not be effective due to the activities of N-cadherin. The DNA microarray chips used in this study were purchased from Affymetrix, HG-U133 Plus 2.0 Arrays. It is a single array, cartridge with greater than 54,000 probe sets are used to analyze the expression level of more than 47,000 transcripts and variants, including approximately 38,500 well characterized human genes. RNA was generated from LNCAP, LNCAP C1, LNCAP C2 cell lines, and LAPC9AI AI early passage xenografts. Full chip service including hybridization, scanning, and data extraction was done with analysis by “R” software. Comparison was done between LNCAP C1 vs C2 and LAPC9AI early passage xenografts, (looking at genes up regulated). Expression was based on statistically significant p and q values. In addition, the genes of interest were also statistically significant against prostate cancer published arrays. 100 up regulated genes of interest were selected.

TABLE 3
Genes upregulated in prostate cancer.

| | REPRESENTATIVE | |
|---|---|---|
| | PUBLIC | |
| | ID/GenBank | |
| GENE SYMBOL | Accession No. | Full Name |
| BG149337 | BG149337 | No gene listed |
| CDH2 | M34064 | cadherin 2, type 1, N-cadherin (neuronal) |
| AADAC | NM_001086 | arylacetamide deacetylase (esterase) |
| DNER | BF059512 | delta/notch-like EGF repeat containing |
| AI861874 | AI861874 | No gene listed |
| SLFN11 | AW003459 | schlafen family member 11 |
| C110rf41 | H08993 | chromosome 11 open reading frame 41 |
| RGS7 | AF493931 | regulator of G-protein signaling 7 |
| KIF21B | NM_017596 | kinesin family member 21B |
| LOC404266 | BC010732 | hypothetical LOC40266 |
| LY6K | BC001291 | lymphocyte antigen 6 complex, locus K |
| SGOL1 | AK024292 | Shugoshin-like 1 (S. pombe), mRNA (cDNA clone IMAGE: 3861301) |
| SIRPD | AL049634 | signal-regulatory protein delta |
| HOXD3 | Y09980 | homeobox D3 |
| CD70 | NM_001252 | CD70 molecule |
| ESM1 | NM_007036 | endothelial cell-specific molecule 1 |
| BE465829 | BE465829 | No gene listed |
| CD83 | NM_004233 | CD83 molecule |
| ICOSLG | AI659611 | inducible T-cell co-stimulator ligand |
| PAX8 | BE465829 | paired box 8 |
| THBS2 | NM_003247 | thrombospondin 2 |
| EMR1 | NM_001974 | egf-like module containing, mucin-like, hormone receptor-like 1 |
| BF513384 | BF513384 | No gene listed |
| UPP1 | NM_003364 | uridine phosphorylase 1 |
| TNF | NM_000594 | tumor necrosis factor (TNF superfamily, member 2) |
| FOXD1 | NM_004472 | forkhead box D1 |
| SIRPB1 | BC025286 | signal-regulatory protein beta 1 |
| RTBDN | BC005063 | Retbindin |
| NMNAT2 | NM_170706 | nicotinamide nucleotide adenylyltransferase 2 |
| BF344237 | BF344237 | No gene listed |
| FSD1 | NM_024333 | fibronectin type III and SPRY domain containing 1 |
| ARL11 | NM_138450 | ADP-ribosylation factor-like 11 |
| MY010 | AI561354 | myosin X |
| AI703496 | AI703496 | No gene listed |
| RHEBL1 | NM_144593 | Ras homolog enriched in brain like 1 |
| IGSF10 | AF087980 | immunoglobulin superfamily, member 10 |
| LRRC8C | BE877357 | leucine rich repeat containing 8 family, member C |
| PAX6 | NM_000280 | paired box 6 |
| HERC5 | NM_016323 | hect domain and RLD 5 |
| BF512556 | BF512556 | No gene listed |
| CEMP1 | BG260181 | Cementum protein 1, mRNA (cDNA clone MGC: 182028 |
| | | IMAGE: 9056853) |
| CKAP2L | AW088063 | cytoskeleton associated protein 2-like |
| AA167270 | AA167270 | No gene listed |
| CCNO | BC004877 | cyclin 0 |
| PHACTR1 | AW054711 | phosphatase and actin regulator 1 |
| EN1 | NM_001426 | engrailed hom eo box 1 |
| FANCA | AW083279 | Fanconi anemia, complementation group A |
| ELAVL2 | AL161628 | ELAV (embryonic lethal, abnormal vision, Drosophila)-like 2 (Hu |
| | | antigen B) |
| C6orf141 | NM_153344 | chromosome 6 open reading frame 141 |
| N6-RE4 | BU587810 | transcription factor NF-E4 |
| AW976431 | AW976431 | No gene listed |
| AI926924 | AI926924 | No gene listed |
| BE877420 | BE877420 | No gene listed |
| GPR39 | AV717094 | G protein-coupled receptor 39 |
| EPHB2 | L41939 | EPH receptor B2 |
| TMEM166 | 227828_s_at | transmembrane protein 166, also known as FLJ13391 |
| NLRP1 | AF229062 | NLR family, pyrin domain containing 1 |
| U66061 | U66061 | protease, serine, 1 (trypsin 1) /// trypsinogen C |
| AI651969 | AI651969 | No gene listed |
| DDX12 | AI983033 | DEAD/H (Asp-Glu-Ala-Asp/His) box polypeptide 11 (CHL 1-like |
| | | helicase homolog, S. cerevisiae) |
| | | //DEAD/H (Asp-Glu-Ala-Asp/His) box polypeptide 12 (CHL 1-like |
| | | helicase homolog, S. cerevisiae) |
| FLJ35409 | BM759658 | FLJ35409 protein |
| BF510692 | BF510692 | No gene listed |
| SRGAP1 | AK023899 | SLIT-ROBO Rho GTPase activating protein 1 |
| SLC2A6 | NM_017585 | solute carrier family 2 (facilitated glucose transporter), member 6 |
| MMP1 | NM_016234 | acyl-CoA synthetase long-chain family member 5 |
| MMP13 | NM_002427 | matrix metallopeptidase 13 (collagenase 3) |
| RGS17 | NM_012419 | regulator of G-protein signaling 17 |
| GALNT6 | NM_007210 | UDP-N-acetyl-alpha-D-galactosamine:polypeptide N- |
| | | acetylgalactosaminyltransferase 6 (GalNAc-T6) |
| AI810767 | AI810767 | No gene listed |
| CTHRC1 | AA584310 | collagen triple helix repeats containing 1 |
| OTOA | NM_170664 | otoancorin pseudogene /// otoancorin |
| TTK | NM_003318 | TTK protein kinase |
| ENTPD4 /// LOXL2 | BE251211 | ectonucleoside triphosphate diphosphohydrolase 4 /// lysyl oxidase- |
| | | like 2 |
| ODZ3 | NM_018104 | odz, odd Ozlten-m homolog 3 (Drosophila) |
| SPAG4 | NM_003116 | sperm associated antigen 4 |
| FSCN1 | BC004908 | fascin homolog 1, actin-bundling protein (Strongylocentrotus |
| | | purpuratus) |
| MGC24039 | NM_144973 | DENN/MADD domain containing 5B (DENND5B) |
| PSIP1 | NM_004682 | PC4 and SFRS1 interacting protein 1 |
| HOXB3 | AW510657 | homeobox B3 |
| PLAUR | U08839 | plasminogen activator, urokinase receptor |
| SLC17A7 | NM_020309 | solute carrier family 17 (sodium-dependent inorganic phosphate |
| | | cotransporter), member 7 |
| RPGR | NM_000328 | retinitis pigmentosa GTPase regulator |
| WSCD1 | BF115148 | WSC domain containing 1 |
| POU3F1 | NM_002699 | POUclass 3 homeobox 1 |
| RBMS2 | AW205585 | RNA binding motif, single stranded interacting protein 2 |
| QSOX2 | AW873348 | quiescin 06 sulfhydryl oxidase 2 |
| SAMHD1 | 205296_at | SAM domain and HD domain 1 |
| LAMP3 | NM_014398 | lysosomal-associated membrane protein 3 |
| B3GNT7 | CA503291 | UDP-GlcNAc:betaGal beta-1,3-N-acetylglucosaminyltransferase 7 |
| INTS4 | NM_033547 | integrator complex subunit 4 |
| LMNB1 | NM_005573 | lamin B1 |
| RAGE | NM_014226 | renal tumor antigen |
| GPSM2 | AW195581 | G-protein signaling modulator 2 (AGS3-like, C. elegans) |
| BG026194 | BG026194 | No gene listed |
| LOC100132352 | BF056548 | PREDICTED: Homo sapiens similar to hCG1989297, transcript |
| | | variant 1 (LOC100132352), mRNA |
| AV734843 | AV734843 | No gene listed |
| FOXA2 | AB028021 | forkhead box A2 |
| TLE4 | NM_007005 | transducin-like enhancer of split 4 (E(sp1) homolog, Drosophila) |
| AW301235 | AW301235 | No gene listed |
| DHX35 | NM_021931 | DEAH (Asp-Glu-Ala-His) box polypeptide 35 |

TABLE 4
Subset of upregulated genes from Table 3.

| Gene symbol | Public ID | Full name |
|---|---|---|
| AA167270 | AA167270 | No gene listed |
| AI651969 | AI651969 | No gene listed |
| AI703496 | AI703496 | No gene listed |
| AI861874 | AI861874 | No gene listed |
| AV734843 | AV734843 | No gene listed |
| AW301235 | AW301235 | No gene listed |
| BF344237 | BF344237 | No gene listed |
| BF512556 | BF512556 | No gene listed |
| BF513384 | BF513384 | No gene listed |
| BG026194 | BG026194 | No gene listed |
| C6orf141 | NM_153344 | chromosome 6 open reading frame 141 |
| CD70 | NM_001252 | CD70 molecule |
| CD83 | NM_004233 | CD83 molecule |
| CDH2 | M34064 | cadherin 2, type 1, N-cadherin (neuronal) |
| DHX35 | NM_021931 | DEAH (Asp-Glu-Ala-His) box polypeptide 35 |
| ELAVL2 | AL161628 | ELAV (embryonic lethal, abnormal vision, Drosophila)- |
| | | like 2 (Hu antigen B) |
| EMR1 | NM_001974 | egf-like module containing, mucin-like, hormone receptor- |
| | | like 1 |
| EN1 | NM_001426 | engrailed homeobox 1 |
| ENTPD4 /// | BE251211 | ectonucleoside triphosphate diphosphohydrolase 4 /// lysyl |
| LOXL2 | | oxidase-like 2 |
| EPHB2 | L41939 | EPH receptor B2 |
| ESM1 | NM_007036 | endothelial cell-specific molecule 1 |
| FOXD1 | NM_004472 | forkhead box D1 |
| ICOSLG | AI659611 | inducible T-cell co-stimulator ligand |
| INTS4 | NM_033547 | integrator complex subunit 4 |
| LMNB1 | NM_005573 | lamin B1 |
| LOC404266 | BC010732 | hypothetical LOC404266 |
| LY6K | BC001291 | lymphocyte antigen 6 complex, locus K |
| MMP1 | NM_016234 | acyl-CoA synthetase long-chain family member 5 |
| MMP13 | NM_002427 | matrix metallopeptidase 13 (collagenase 3) |
| MYO10 | AI561354 | myosin X |
| N6-RE4 | BU587810 | transcription factor NF-E4 |
| NLRP1 | AF229062 | NLR family, pyrin domain containing 1 |
| ODZ3 | NM_018104 | odz, odd Oz/ten-m homolog 3 (Drosophila) |
| OTOA | NM_170664 | otoancorin pseudogene /// otoancorin |
| PLAUR | U08839 | plasminogen activator, urokinase receptor |
| POU3F1 | NM_002699 | POU class 3 homeobox 1 |
| PSIP1 | NM_004682 | PC4 and SFRS1 interacting protein 1 |
| RGS7 | AF493931 | regulator of G-protein signaling 7 |
| RTBDN | BC005063 | retbindin |
| SIRPD | AL049634 | signal-regulatory protein delta |
| SLC17A7 | NM_020309 | solute carrier family 17 (sodium-dependent inorganic |
| | | phosphate cotransporter), member 7 |
| SRGAP1 | AK023899 | SLIT-ROBO Rho GTPase activating protein 1 |
| THBS2 | NM_003247 | thrombospondin 2 |
| WSCD1 | BF115148 | WSC domain containing 1 |

It is understood that the examples and embodiments described herein are for illustrative purposes only and that various modifications or changes in light thereof will be suggested to persons skilled in the art and are to be included within the spirit and purview of this application and scope of the appended claims. All publications, including those the publications associated with Genbank accession numbers and particularly the disclosed nucleic acid and polypeptide sequences therein, patents, and patent applications cited herein are hereby incorporated by reference in their entirety for all purposes.

What is claimed is:

1. A method of diagnosing a cancer in a subject, the method comprising the steps of:
(a) analyzing a tissue sample from the subject with an assay that specifically detects at least one marker that is a downstream target of N-cadherin, wherein the at least one marker is selected from the markers listed in Table 3 or Table 4; and
(b) determining whether or not expression of the at least one marker is altered in the tissue sample; thereby providing a diagnosis for the cancer.

2. The method of claim 1, wherein the assay detects nucleic acid and is mass spectroscopy, PCR, microarray hybridization, thermal cycle sequencing, capillary array sequencing, or solid phase sequencing.

3. The method of claim 1, wherein the assay detects protein and is ELISA, Western blotting, flow cytometry, immunofluorescence, immunohistochemistry, or mass spectroscopy.

4. The method of claim 1, wherein the assay comprises a reagent that binds to a nucleic acid.

5. The method of claim 4, wherein the reagent is a nucleic acid.

6. The method of claim 5, wherein the reagent is an oligonucleotide.

7. The method of claim 6, wherein the reagent is an RT-PCR primer set.

8. The method of claim 1, wherein the assay comprises a reagent that binds to a protein.

9. The method of claim 8, wherein the reagent is an antibody.

10. The method of claim 1, wherein the cancer is an N-cadherin-expressing cancer.

11. The method of claim 10, wherein the cancer is prostate cancer.

12. The method of claim 1, wherein the at least one marker is from Table 4.

13. The method of claim 1, wherein the tissue sample is a metastatic cancer tissue sample.

14. The method of claim 1, wherein the tissue sample is prostate tissue.

15. The method of claim 1, wherein step (b) comprises determining whether or not the at least one marker is overexpressed in the tissue sample; thereby providing the diagnosis for the cancer.

16. A method of providing a prognosis for a cancer in a subject, the method comprising the steps of:
(a) analyzing a tissue sample from the subject with an assay that specifically detects at least one marker that is a downstream target of N-cadherin, wherein the at least one marker is selected from the markers listed in Table 3 or Table 4; and
(b) determining whether or not expression of the at least one marker is altered in the tissue sample; thereby providing a prognosis for the cancer.

17. The method of claim 16, wherein the assay detects nucleic acid and is mass spectroscopy, PCR, microarray hybridization, thermal cycle sequencing, capillary array sequencing, or solid phase sequencing.

18. The method of claim 16, wherein the assay detects protein and is ELISA, Western blotting, flow cytometry, immunofluorescence, immunohistochemistry, or mass spectroscopy.

19. The method of claim 16, wherein the assay comprises a reagent that binds to a nucleic acid.

20. The method of claim 19, wherein the reagent is a nucleic acid.

21. The method of claim 20, wherein the reagent is an oligonucleotide.

22. The method of claim 21, wherein the reagent is an RT-PCR primer set.

23. The method of claim 16, wherein the assay comprises a reagent that binds to a protein.

24. The method of claim 23, wherein the reagent is an antibody.

25. The method of claim 16, wherein the cancer is an N-cadherin-expressing cancer.

26. The method of claim 25, wherein the cancer is prostate cancer.

27. The method of claim 16, wherein the at least one marker is from Table 4.

28. The method of claim 16, wherein the tissue sample is a metastatic cancer tissue sample.

29. The method of claim 16, wherein the tissue sample is prostate tissue.

30. The method of claim 16, wherein step (b) comprises determining whether or not the at least one marker is overexpressed in the tissue sample; thereby providing the prognosis for the cancer.
